(19) United States
(12) Patent Application Publication
(10) Pub. No.: US 2025/0062030 A1
(43) Pub. Date: Feb. 20, 2025

(54) SYSTEM AND METHOD FOR ADAPTIVE GENERATION OF GRAPHICAL DATA OF PREDICTED DIAGNOSES

(71) Applicant: Roche Diabetes Care, Inc., Indianapolis, IN (US)

(72) Inventors: Samer Dajani, Carmel, IN (US); Chinmay Manohar, Carmel, IN (US)

(73) Assignee: Roche Diabetes Care, Inc., Indianapolis, IN (US)

(21) Appl. No.: 18/934,320

(22) Filed: Nov. 1, 2024

Related U.S. Application Data
(63) Continuation of application No. PCT/US2023/066475, filed on May 2, 2023.
(60) Provisional application No. 63/364,518, filed on May 11, 2022.

(51) Int. Cl. G16H 50/20; G06F 3/04847; G06T 11/20; G16H 10/60

(52) U.S. Cl. CPC G16H 50/20; G06T 11/206; G16H 10/60; G06F 3/04847; G06T 2200/24; G06T 2210/41

(57) ABSTRACT

A method for generating a user interface of predicted disease progressions includes receiving medical data corresponding to patient visits to a healthcare provider, generating a diagnosis for the patient based on the medical data, and generating a predicted diagnosis for a future condition of the patient based upon the diagnosis, and a predictive model. The method further includes generating a timeline view of a diagnosis in the current patient visit and the predicted diagnosis. The graphical element of the diagnosis and the predicted diagnosis both include a graphical indicator of a diagnosis and at least one graphical sub-element of a physiological parameter relevant to the diagnosis. The method further includes generating a graphical connector between the graphical elements to indicate progression of time between a first time of the current patient visit and a second time.

### CLAIM OF PRIORITY AND CROSS-REFERENCE TO RELATED APPLICATIONS

This application claims priority to Patent Cooperation Treaty Application No. PCT/US2023/066475, which is entitled “SYSTEM AND METHOD FOR ADAPTIVE GENERATION OF GRAPHICAL DATA OF PREDICTED DIAGNOSES,” and was filed on May 2, 2023, the entire contents of which are hereby incorporated herein by reference. This application claims further priority to U.S. Provisional Patent Application No. 63/364,518, which is entitled “SYSTEM AND METHOD FOR ADAPTIVE GENERATION OF GRAPHICAL DATA OF PREDICTED DIAGNOSES,” and was filed on May 11, 2022, the entire contents of which are hereby incorporated herein by reference. This application cross-references Patent Cooperation Treaty Application No. PCT/US2023/066473, which is entitled “SYSTEM AND METHOD FOR ADAPTIVE GENERATION OF GRAPHICAL DATA OF A TREATMENT HISTORY” and was filed on May 2, 2023, the entire contents of which are hereby incorporated herein by reference. This application further cross-references U.S. Provisional Patent Application No. 63/364,517, which is entitled “SYSTEM AND METHOD FOR ADAPTIVE GENERATION OF GRAPHICAL DATA OF A TREATMENT HISTORY” and was filed on May 11, 2022, the entire contents of which are hereby incorporated herein by reference.

### BACKGROUND

Persons with Diabetes (PwDs), and particularly those with type 2 diabetes (T2D), most often receive treatment from Primary Care Physicians (PCPs) such as General Practitioners (GPs) or Family Medicine Physicians (FMs) healthcare providers. Primary care physicians are often overwhelmed due to high patient volumes, since they not only see patients with T2DM but also those with many other chronic conditions. With typical appointments lasting less than 15 minutes, there is a tremendous cognitive load on the PCPs when it comes to disease management, and optimal therapy recommendations. These factors lead to the phenomenon of clinical inertia, which is the delay caused in appropriate intensification of therapies for better disease management. Clinical inertia, in turn, leads to high health-economic costs and negative quality of life implications for PwDs.

Therefore, there is a need for Clinical Decision Support (CDS) tools that aid PCPs and other healthcare providers (HCPs) in selecting the appropriate therapies for people with type 2 diabetes. When integrated within their clinical workflow, a CDS tool that takes into account patient characteristics, can help HCPs in making better personalized therapy decisions improving clinical, patient-reported and economical outcomes. While the medical field has recognized clinical guidelines for diabetes treatment from bodies such as the American Diabetes Association (ADA) and the European Association for the Study of Diabetes (EASD), even these clinical guidelines for therapy transitions in T2D can be cumbersome for a PCP to follow or apply given the volume of patient data. Given the chronic nature of diabetes and many of its comorbidities, healthcare providers also have a need to review and share potential prognoses with the PwD to assess different treatment options and to help educate the PwD about disease progression. While statistical models exist that can provide help with predicting disease trajectory, an HCP with limited patient time in each visit may not have the opportunity to consult such models to provide the PwD with different prognoses in a rigorous manner. Consequently, improvements to systems and methods that provide clinical information and CDS to PCPs and other HCPs in an efficient manner that reduce cognitive load and enables an HCP to easily review different predictions for disease progression would be beneficial.

### SUMMARY

An adaptive clinical decision support user interface (UI) identifies and generates graphical data for a user interface with one or more predicted disease progressions for a patient based on a current patient diagnosis, optionally one or more treatment options, and a predictive model. The model based predictive approach enables a healthcare provider (HCP) at the point of care to quickly visualize and discuss therapy options with the patients by not only displaying potential disease progressions for different prescribed treatment options, but also add other contextual elements such expected durations for each stage and likely outcomes both health and economics wise and likelihood of success for each of these trajectories.

In one embodiment, a method for generating a user interface of a treatment history for a patient has been developed. The method includes receiving, with a processor, medical data for the patient, the medical data corresponding to at least one patient visit to a healthcare provider, generating, with the processor, a first diagnosis for the patient during a current patient visit based on the medical data, generating, with the processor, a first predicted diagnosis for a future condition of the patient based at least in part upon the first diagnosis and a predictive model stored in a memory operatively connected to the processor, and generating, with the processor, graphical data corresponding to a timeline view of the current patient visit and the first predicted diagnosis. The generating of the graphical data further includes generating a first graphical element corresponding to the first diagnosis, the first graphical element further including a graphical indicator of the first diagnosis and at least one graphical sub-element, the at least one graphical sub-element being relevant to a physiological parameter selected from the medical data, the physiological parameter being related to the first diagnosis, and generating a second graphical element corresponding to the first predicted diagnosis, the second graphical element further including a graphical indicator of the first predicted diagnosis and at least one graphical sub-element, the at least one graphical sub-element being relevant to a physiological parameter related to the first predicted diagnosis. The method further includes generating a first graphical connector between the first graphical element and the second graphical element, the first graphical connector indicating a progression of time between a first time of the current patient visit and a second time in the timeline view.

In another embodiment, a computing system that is configured to generate a user interface of a treatment history for a patient includes a memory and a processor operatively connected to the memory. The memory is configured to store medical data for the patient, the medical data corresponding to at least one patient visit to a healthcare provider, a predictive model, and stored program instructions. The processor is configured to execute the stored program instructions to generate a first diagnosis for the patient during a current patient visit based on the medical data, generate a first predicted diagnosis for a future condition of the patient based at least in part upon the first diagnosis and the predictive model, and generate graphical data corresponding to a timeline view of the current patient visit and the first predicted diagnosis. The processor being further configured to generate a first graphical element corresponding to the first diagnosis, the first graphical element further including a graphical indicator of the first diagnosis and at least one graphical sub-element, the at least one graphical sub-element being relevant to a physiological parameter selected from the medical data, the physiological parameter being related to the first diagnosis, and generate a second graphical element corresponding to the first predicted diagnosis, the second graphical element further including a graphical indicator of the first predicted diagnosis and at least one graphical sub-element, the at least one graphical sub-element being relevant to a physiological parameter related to the first predicted diagnosis. The processor is further configured to generate a first graphical connector between the first graphical element and the second graphical element, the first graphical connector indicating a progression of time between a first time of the current patient visit and a second time in the timeline view.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a schematic diagram of a computer system that is configured to generate graphical data of a medical timeline for a patient in an adaptive user interface for a healthcare provider including graphical elements that depict predicted diagnoses that are identified using a predictive model.

FIG. 2 is a block diagram of a method of operation for the system of FIG. 1.

FIG. 3 is a depiction of a timeline view that incorporates graphical data of a diagnosis for a current patient visit, a prescribed treatment option for the patient, and one or more predicted diagnoses for a future patient visit.

FIG. 4 is a depiction of the timeline view of FIG. 3 extended in time to include additional predicted diagnoses for a second future patient visit.

FIG. 5 is a depiction of the timeline view of FIG. 3 with an adjusted ranking threshold that displays predicted diagnoses having a highest probability and second highest probability as identified in the predictive model.

### DETAILED DESCRIPTION

These and other advantages, effects, features and objects are better understood from the following description. In the description, reference is made to the accompanying drawings, which form a part hereof and in which there is shown by way of illustration, not limitation, embodiments of the inventive concept. Corresponding reference numbers indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, and features falling within the spirit and scope thereof as defined by the embodiments described herein and the embodiments below. Reference should therefore be made to the embodiments described herein and embodiments below for interpreting the scope of the inventive concept. As such, it should be noted that the embodiments described herein may have advantages, effects, and features useful in solving other problems.

The devices, systems and methods now will be described more fully hereinafter with reference to the accompanying drawings, in which some, but not all embodiments of the inventive concept are shown. Indeed, the devices, systems and methods may be embodied in many different forms and should not be construed as limited to the embodiments set forth herein; rather, these embodiments are provided so that this disclosure will satisfy applicable legal requirements.

Likewise, many modifications and other embodiments of the devices, systems and methods described herein will come to mind to one of skill in the art to which the disclosure pertains having the benefit of the teachings presented in the foregoing descriptions and the associated drawings. Therefore, it is to be understood that the devices, systems and methods are not to be limited to the specific embodiments disclosed and that modifications and other embodiments are intended to be included within the scope of the embodiments. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may refer both to a situation in which, besides B, no other element is present in A (i.e., a situation in which A solely and exclusively consists of B) or to a situation in which, besides B, one or more further elements are present in A, such as element C, elements C and D, or even further elements.

As used herein, the term Person with Diabetes (PwD) refers to a patient who is diagnosed with or is at-risk for being diagnosed with one or more forms of diabetes including of pre-diabetes, type 1 diabetes, type 2 diabetes, gestational diabetes, as well as one or more comorbidities that are associated with diabetes. In the specific embodiments described herein, the PwD is a patient of a healthcare provider (HCP), and references to the PwD and a patient are used interchangeably herein. While the specific embodiments that are described herein are directed to improving the user interface of the patient treatment history for HCPs who treat PwDs, the systems and methods described herein are not limited to the treatment of PwDs and may be used to implement improved user interfaces for the treatment of other diseases and medical conditions, and particularly chronic medical conditions that require long term treatment.

As used herein, the term “physiological parameter” refers to any quantifiable aspect of a PwD's physiology that is measured as part of providing medical data to diagnose a new medical condition or to track the state of a previously diagnosed medical condition. A non-limiting list of physiological parameters that are of interest to the treatment of diabetes and diabetes comorbidities includes body mass index (BMI), blood pressure (BP), blood glucose, glycosylated hemoglobin (HbA1c), blood ketones, and estimated glomerular filtration rate (eGFR).

As used herein, the term “medical data” refers to include both medical diagnostic data and medical treatment data. The medical diagnostic data include identifications of prior diagnoses, diagnostic test results, and records of prior and current physiological parameter values for the PwD. The medical diagnostic data optionally include relevant genetic data, phenotype data, demographic data, and socio-economic data pertaining to the PwD. The medical treatment data include records of previously prescribed medications or other medical treatments that have been prescribed to the PwD during previous patient visits. During a current patient visit, a clinical decision support system is configured to generate one or more prescribed treatments for a PwD based on the medical data and an HCP can adopt a prescribed treatment or manually select a different course of action for the PwD.

As used herein, the term “prescribed treatment” refers to any medical diagnostic tests, medical diagnostic or prognostic algorithms, medical procedures, medical therapies, medications, diet and lifestyle modification, or other recommended course of action that the HCP issues or has the option to issue to the PwD during the treatment history for the PwD. In particular, regarding medications the term “prescribed” here encompasses both over-the-counter and prescription medications.

As used herein, the term “graphics data” refers to any form of encoded data that a computing device uses to generate a visually perceptible output including text, geometry, pictures, icons, textures, and the like using a display device, printer, or other output device. Different forms of graphics data include both static image data and moving images such as animations and video. Examples of graphics data include rasterized image data, vector graphics data, procedural graphics data, and combinations thereof. Examples of rasterized image data include graphics data that encode an array of pixel values in an image, where a display device generates an output image formed from the array of pixel values. Rasterized image data may be compressed using the JPEG, PNG, WEBP, or other suitable compression formats for static images and using video compression codecs such as h.264, h.265, VP9, AV1, or other suitable compression formats for video or animations. Examples of vector graphics include graphics data that encode declarative parameters that describe the shapes, colors, arrangements, and other details of an image that a computing device processes to reproduce an image, and examples of vector graphics include the scalable vector graphics (SVG), graphics generated from cascading style sheet (CSS) documents, portable document format (PDF), and other suitable vector graphics formats. Procedural graphics data includes data encoded as imperative command data that a processor executes to generate graphics data in a dynamic manner. Examples of procedural graphics data include encoded command parameters to control JavaScript, WebAssembly, WebGL, or another scripting language to draw graphics as part of the HTML <canvas> element used in publicly available web browsers, or data encoded in the Postscript language that a computing device renders using a Postscript rendering engine. Furthermore, markup language formats such as the hypertext markup language (HTML), extensible markup language (XML), or the suitable markup languages may be used to format the arrangement of one or more sets of graphics data that form graphical elements to generate the timeline views and other graphics described herein. In some configurations, a single computing system generates graphics data and performs the process of rendering the graphics data to a display device for human users to view the graphics. As described in further detail below, in other configurations a first computing system generates the graphics data and transmits the graphics data to one or more computing systems that perform the task of rendering the graphics to one or more display devices to enable one or more human users to view the graphics.

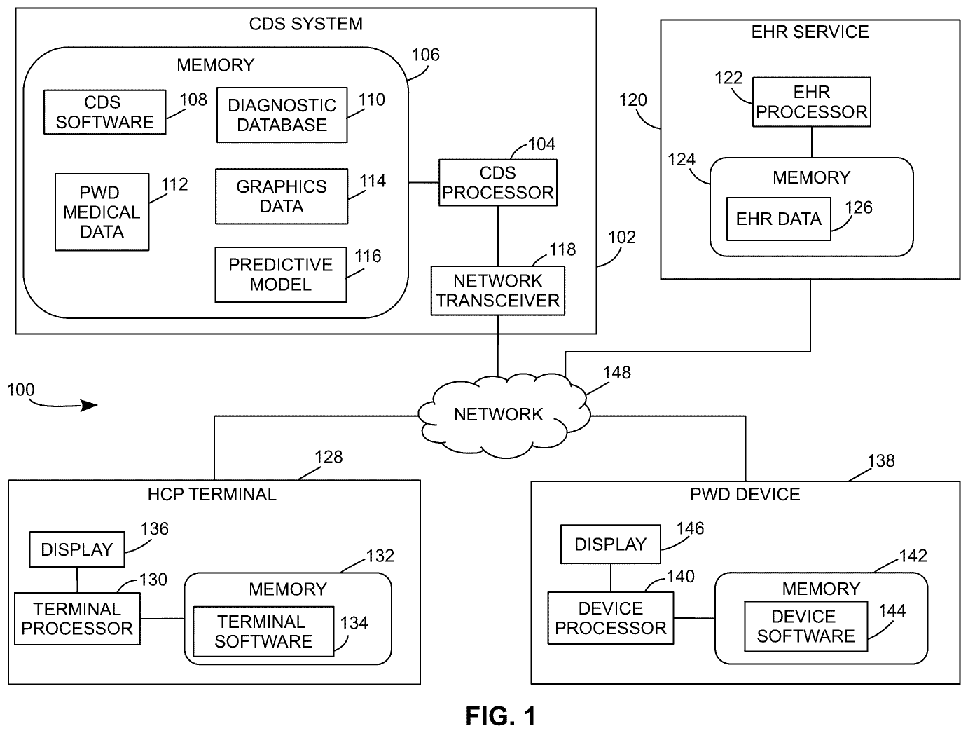

FIG. 1 depicts a system 100 that provides clinical decision support information to HCPs with the adaptive user interfaces described herein. The system 100 includes a clinical decision support (CDS) system 102, an electronic health record (EHR) service 120, an HCP terminal 128, and an optional PwD device 138. The CDS system 102, EHR Service 120, HCP terminal 128, and PWD device 138 are communicatively connected via a network 148.

The CDS system 102 of FIG. 1 performs the functions described herein utilizing one or more computing devices that include one or more central processing units (CPUs), graphics processing units (GPUs), application specific integrated circuits (ASICs), field programmable gate arrays (FPGAs), other digital logic devices, or combinations thereof that are depicted as the CDS processor 104 in FIG. 1. The CDS processor 104 is operatively connected to a CDS memory 106 and a network transceiver 118. The CDS memory 106 includes one or more volatile data storage devices such as static and dynamic random access memory (RAM) and one or more non-volatile data storage devices such as magnetic, solid state, and optical storage drives. During operation, the CDS processor 104 reads and writes data to the CDS memory 106 to execute stored program instructions and to store data including medical data received from the HCP terminal 128 and the EHR service 120 and generated graphics data. The CDS processor 104 operates the network transceiver 118, which is a wired or wireless network interface controller that transmits and receives data via the network 148, to receive medical data and other commands from the HCP terminal 128, transmit and receive EHR data with the EHR service 120, and transmit graphical data of a timeline view user interface of treatment history for the PwD to the HCP terminal 128. In some configurations, the CDS processor 104 also operates the network transceiver 118 to receive medical data directly from the PWD device 138 and to transmit the graphical data of a treatment history for the PwD to the PWD device 138.

In the CDS system 102, the CDS memory 106 stores CDS software 108, a diagnostic database 110, PwD medical data 112, graphics data 114, and a predictive model 116. The CDS software 108 includes stored program instructions that the CDS processor 104 executes to perform the clinical support functions and generate graphical data corresponding to a timeline view of one or more diagnoses for the PwD and one or more prescribed treatments to treat the PwD over a series of patient visits. The CDS software 108 also implements one or more network interfaces, such as web servers or other network servers, to enable the HCP terminal 128 to access and send commands to the CDS system 102 and to enable the CDS system 102 to transmit the generated graphical data described herein to the HCP terminal 128.

In the CDS memory 106, the diagnostic database 110 includes a stored set of logical rules that the CDS system 102 uses to generate diagnoses for the PwD based on the medical data for the PwD. In one specific configuration, the diagnostic database 110 encodes guidelines from the American Diabetic Association (ADA) Standards of Medical Care in Diabetes. Alternative configurations employ different medical guidelines or other algorithms to generate diagnoses and prescribed treatments. While the CDS system 102 uses the diagnostic database 110 to generate graphical elements that display diagnoses and prescribed treatments in an automated manner during operation of the system 100, an HCP optionally uses the HCP terminal 128 to override the suggested diagnosis or course of action recommendations.

In the CDS memory 106, the PwD medical data 112 include any relevant medical data for the PwD during both a current patient visit and historic medical record data including medical data for one or more prior patient visits. The PwD medical data 112 optionally include PwD medical data that the CDS system 102 receives from the EHR service 120 with historic medical data stored in an electronic health record for the PwD as well as data received from external lab tests, home diagnostics such as spot and continuous blood glucose meter devices, and medical data that an HCP provides to the CDS system 102 via the HCP terminal 128 during a patient visit. Furthermore, during operation the CDS system 102 optionally transmits updated medical data for the PwD to the EHR service 120 to reflect updated measurements to physiological parameters, diagnoses, or to record prescribed medications or other medical treatments that the PwD receives during a course of treatment.

In the CDS memory 106, the graphics data 114 include a graphical representation of a timeline view user interface of a treatment history for the PwD over a series of one or more patient visits based on the PwD medical data 112. For example, the graphics data 114 form a timeline view including graphical elements and sub-elements to depict one or more diagnoses and relevant physiological parameters for each diagnosis, prescribed treatments, prescribed medications, diagnostic tests, and graphical connectors that link the graphical elements in the timeline view. In some configurations, the graphics data 114 further include stored text, icons, geometric templates, and other visually perceivable data that the CDS system 102 uses for adaptive generation of the timeline view for the PwD based on the PwD medical data 112.

In the CDS memory 106, the predictive model 116 is embodied as, for example, a stochastic model or a machine-learning model. Examples of stochastic models include a Markov Chain, Markov Decision Process, semi-Markov Chain, or semi-Markov Decision Process. Additional details of these stochastic models as implemented in the CDS system 102 are provided in further detail below. While these predictive models are described in further detail herein as non-limiting examples for illustrative purposes, alternative configurations of the CDS system 102 can utilize predictive models that incorporate different stochastic models or that rely upon machine learning models to generate predictions for the disease progression. For example, artificial neural networks (ANNs) in general, and, in particular, recurrent neural networks such as long short-term memory (LSTM) neural networks may be used to generate disease progression predictions. In general, any predictive model that is known to the art and that is suitable for generating predicted diagnoses may be utilized in the CDS system 102.

As is known in the art, Markov Chains model probabilities for transitions between states, where each state corresponds to a stage in the progression of a disease, based on a current state, where the current state is the currently diagnosed condition of a PwD and optionally other known information about the PwD that associates the PwD with a state in the Markov Chain. The Markov Chain provides probability values for transitioning to different states based on a discrete time increment, which corresponds to the time interval between patient visits in illustrative example of FIG. 1. Additionally, a transition that results in remaining in the current state, which corresponds to the PwD remaining in a stable condition through the next patient visit, is typically one potential outcome of the state transition in the Markov Chain. The Markov Decision Process is a modification of the Markov Chain in which the probability values for transitions to different states are determined based on the current state, as in the Markov Chain, and additionally on a decision or “action”, where the action in the context of the CDS system 102 refers to a prescribed treatment that potentially affects the disease progression and consequently changes the probabilities for transitions to different states in the Markov Decision Model. By contrast, a Markov Chain encodes information for different prescribed treatment options into the present state for the PwD, and as such each prescribed treatment option in addition to the diagnosed condition and other medical data corresponds to one current state in a set of current states for the PwD in the Markov Chain, where the selection of a different prescribed treatment changes the current state and corresponding state transition probabilities for the PwD in the Markov Chain.

The semi-Markov Chain and semi-Markov Decision Process predictive models refer to variants of the Markov Chain and Markov Decision Process models that are described above in which the transition probabilities are further determined using a “sojourn time” parameter that corresponds to the length of time that the PwD has spent in the current state, which is determined based on the medical history data for the PwD. For example, the semi-Markov Chain and semi-Markov Decision Process predictive models treat a patient with newly diagnosed elevated Hba1c levels (zero sojourn time) differently from a patient who has experienced the elevated Hba1c levels for a year, which in turn affects the probability values for the future progression of HbA1c levels or other diagnosed conditions in the predictive model.

During the generation of the predictive model 116 for use with the CDS system 102, the precise probability values for each state transition are derived from empirical data such as epidemiological study data, actuarial tables, insurance claim data, and other sources of clinical data that provide statistical data relevant to the disease progression for diabetes and diabetic comorbidities. The predictive model 116 is generated prior to operation of the CDS system 102 and the system 100 as further described herein.

The EHR service 120 of FIG. 1 provides medical data for the PwD to the CDS system 102 in the form of an EHR. In the illustrative embodiment of FIG. 1, the EHR service is a networked computing service that includes a digital processor 122, a memory 124 that stores the EHR data 126 for the PwD. The EHR data 126 is a digital record that is encoded in a standard format such as the Fast Healthcare Interoperability Resource (FHIR) format, a version of the Health Level Seven International (HL7) format, or another suitable electronic health record format. In the embodiment of FIG. 1, the EHR service 120 operates independently of the CDS system 102, but in an alternative configuration the EHR service 120 and the CDS system 102 may be implemented as a unified system. In practical embodiments, the EHR data 126 for the PwD may include data received from multiple data sources including external diagnostic test services, the HCP who operates the HCP terminal 128, the CDS system 102, and EHR data from other HCPs such as medical specialists who treat the PwD for other medical conditions and comorbidities.

The HCP terminal 128 of FIG. 1 is a desktop or laptop personal computer (PC), tablet, smartphone, or other suitable client computing device of the HCP that includes a terminal processor 130, memory 132, and a display device 136. The HCP uses the HCP terminal 128 to provide medical data to the CDS system 102 and optionally the EHR services 120, and to receive and display graphical data corresponding to a timeline view of at least one patient visit for a PwD as the PwD receives treatment over a series of visits with the HCP. In some configurations, the HCP also uses the HCP terminal 128 to communicate with the PWD device 138 between patient visits or to conduct remote patient visits in situations where the HCP provides telehealth services to the PwD. In the embodiment of FIG. 1, HCP terminal 128 executes stored program instructions in the terminal software 134 stored in the HCP terminal memory 132 to enable the HCP terminal 128 to communicate with the CDS service 102. In one configuration, the terminal software 134 includes operating system and web browser software that acts as a client to one or more web services provided by the CDS system 102, but in alternative configurations the terminal software 134 is another client software program. During operation, the terminal processor 130 executes the terminal software 134 and operates the display device 136 to generate a visible output of the graphics data 114 that the CDS system 102 generates and transmits to the HCP terminal 128. The display device 136 is, for example, a flat panel display screen or other electronic display device, although in some configurations a printer may reproduce the graphical display of the timeline view on paper or another print medium. In some configurations, the display device 136 incorporates a touchscreen interface to enable the HCP to enter data and modify the timeline view as described in further detail below, although in other configurations the HCP terminal 128 incorporates a combination of a mouse, keyboard, voice input device, or other input devices [not shown] to receive HCP input.

The PWD device 138 of FIG. 1 is another desktop or laptop PC, tablet, smartphone, or other suitable client computing device of the PwD that includes a device processor 140, a device memory 142 that stores PwD device software 144, and a display device 146. In some configurations, the PWD device 138 receives physiological parameter data from a monitoring device such as a spot or continuous blood glucose meter, a fitness tracking device such as a smart watch, or other medical device. The PWD device 138 is optionally configured to enable the PwD to conduct a telehealth patient visit with the HCP via the HCP terminal 128 using videoconferencing software and other telehealth software that is otherwise known to the art and is not described in further detail herein. In some configurations, the CDS system 102 transmits generated graphics data for the timeline view user interface of the treatment history for the PwD to the PWD device 138 for display using the display device 146 via the network 148. In another configuration, the HCP terminal 128 re-transmits the graphics data for the timeline view of the treatment history for the PwD to the PWD device 138 during a telehealth patient visit.

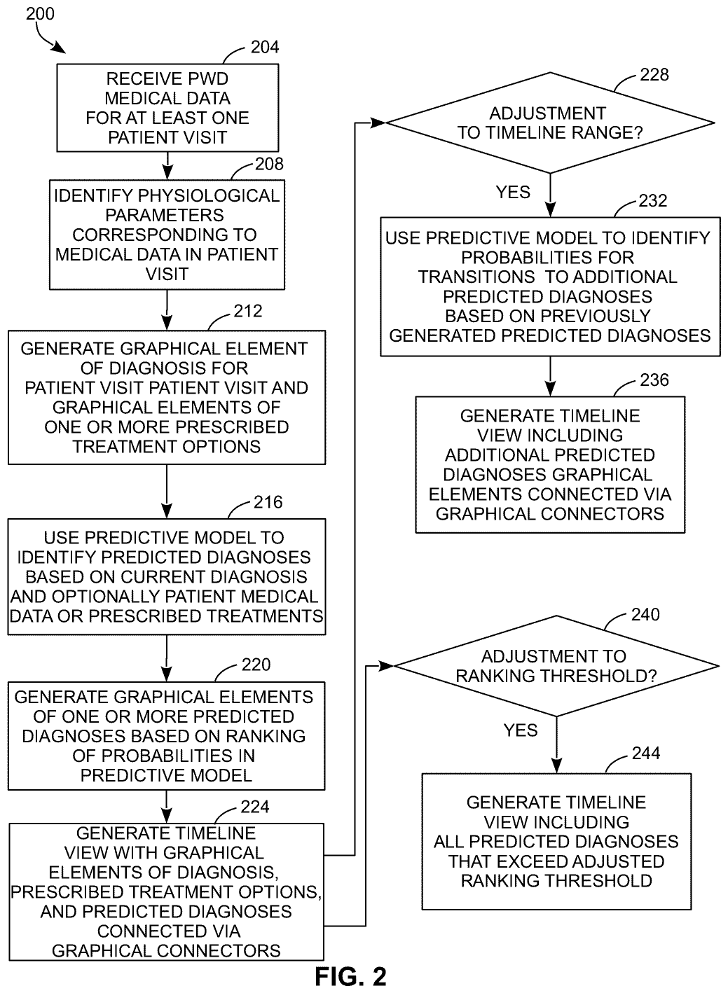

FIG. 2 is a block diagram of a process 200 for the operation of a CDS system to generate a graphical timeline view user interface of a currently diagnosed condition for a PwD, potential prescribed treatment options, and predictions for future disease progression over one or more future patient visits. While the process 200 can be performed at any time, for illustrative purposes the process 200 is described as occurring during a patient visit to generate the graphical timeline view of the current patient visit and one or more predictions for disease prediction. The process 200 is described in conjunction with the system 100 of FIG. 1, and a reference to the process 200 performing a function or action refers to the operation of a processor, such as the CDS processor 104 in CDS system 102, to execute stored program instructions, such as the CDS software 108, to perform the function or action.

The process 200 begins as the CDS system 102 receives PwD medical data for at least one patient visit (block 204). In the system 100, processor 104 in the CDS system 102 receives the medical data from one or more sources including, but not limited to, the EHR service 120, the HCP terminal 128, and, in some instances, the PWD device 138. In particular, the EHR service 120 provides medical data for prior patient visits including prior diagnoses, prescribed medications and medical treatments, a historic record of physiological parameter data measurements for the PwD, and optionally socio-economic and demographic data pertaining to the PwD. During a patient visit, the HCP terminal 128 optionally transmits physiological parameter and other medical data to the CDS system 102 based on manual input from the HCP, from automatically uploaded physiological parameters generated by medical testing devices such as blood glucose meters, or both. Additionally, the CDS system 102 may receive medical data regarding blood tests or other diagnostic tests that the PwD receives at an external diagnostic laboratory prior to the patient visit either directly from a computing system of the diagnostic laboratory, via the EHR service 120, or from the HCP terminal 128. As described above, the CDS system 102 stores the received PwD medical data 112 in the CDS memory 106.

The process 200 continues as the CDS system 102 identifies physiological parameters that have the greatest relevance to a diagnosis during each patient visit (block 208). In the embodiment of FIG. 1, the CDS processor 104 uses the diagnostic database 110 to identify the physiological parameters in the PwD medical data 112 that have the greatest relevance to each diagnosis in a series of one or more patient visits. For example, if a patient visit includes diagnoses that a PwD is both obese and has uncontrolled HbA1c, which indicate the onset of type 2 diabetes, then the CDS processor 104 uses the diagnostic database 110 to identify the physiological parameters that have the highest relevance to these diagnoses, such as body mass index (BMI) related to obesity and the measured HbA1c level for the PwD. In many instances, the PwD medical data 112 include physiological parameters and other information that are not directly relevant to a diagnosis, and the CDS processor 104 filters these data from the timeline view, although an HCP may of course access the full PwD medical data 112 via a traditional user interface if desired.

The process 200 continues as the CDS system 102 generates a graphical element of a diagnosed condition for the PwD during the current patient visit and one or more graphical elements of potential prescribed treatment options (block 212). The graphical element of the diagnosed condition further includes and at least one graphical sub-element for one or more identified physiological parameters that are relevant to the diagnosis for the PwD. Graphical elements and graphical sub-elements provide graphical indicators of the physiological parameter data that led to the diagnosis and stated course of treatment. A graphical indicator refers to any type of graphics data in the timeline view that conveys specific information about the medical data for the PwD, a prescribed treatment for the PwD, or a recommendation for a prescribed treatment for the PwD. A graphical sub-element is a type of graphical element that is subordinate to another graphical element in the timeline view, and the CDS system 102 generates the graphics data for the graphical sub-elements within the borders of a parent graphical element or otherwise associates each graphical sub-element with a parent graphical element. The graphical elements and sub-elements provide a clear indication of the relationship between a graphical element for a patient visit and one or more graphical sub-elements that are related to the patient visit. The CDS system 102 identifies potential prescribed treatments for the diagnosed condition using the diagnostic database 110 and generates a graphical element for each potential prescribed treatment option that identifies the prescribed treatment, such as a type of medication, diagnostic test, or other medical therapy, and optionally includes additional useful information pertaining to the prescribed treatment for the HCP.

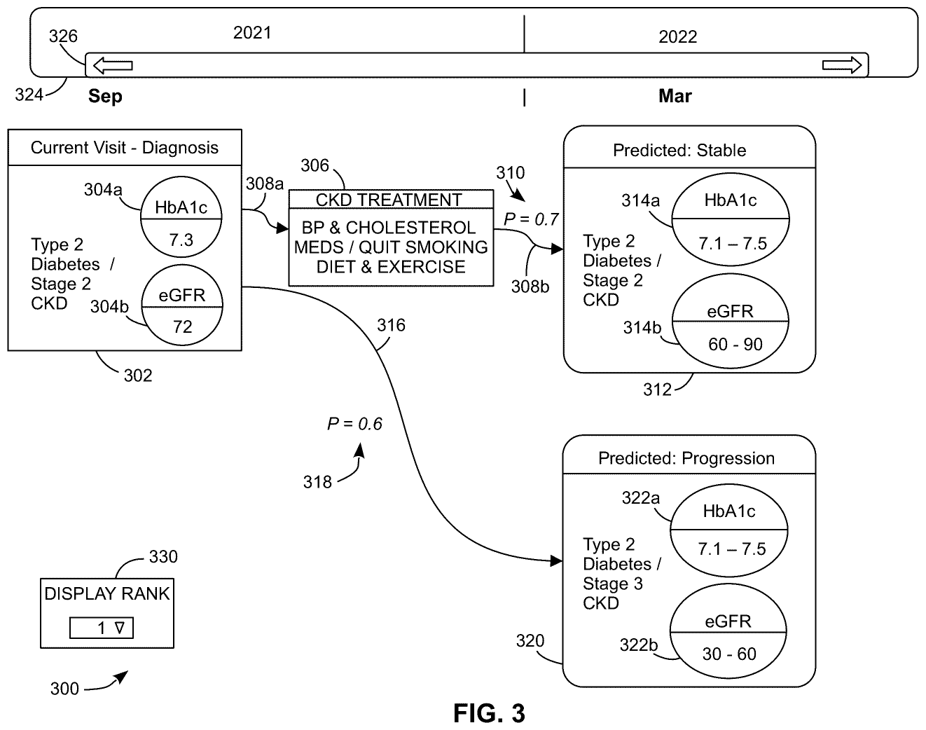

FIG. 3 depicts one example of a timeline view 300 that includes a graphical element 302 that depicts the diagnosis for a PwD during a current patient visit and a graphical element 306 that depicts a treatment option for the PwD. In the specific example of the timeline view 300, the diagnosis for the current visit includes a diagnosis of chronic kidney disease (CKD) in a person with type 2 diabetes. CKD is a known comorbidity for PwDs that is also known to be a progressive condition.

In the timeline view 300, the graphical element 302 is depicted as a rectangular graphical element that includes a graphical indicator of the first diagnosis, which in the example of FIG. 3 is a text label indicating the diagnosis of Type 2 Diabetes and Stage 2 CKD. The graphical element 302 further includes graphical sub-elements 304a and 304b that are located inside of the graphical element 302. The graphical sub-element 304a is depicted as a circle that includes a graphical indicator of a physiological parameter, HbA1c in an upper semi-circle, and a measured value of the HbA1c for the PwD in a lower semi-circle. The HbA1c physiological parameter is related to the Type 2 Diabetes diagnosis. Similarly, the graphical sub-element 304b is depicted as another circle that includes a graphical indicator of another physiological parameter, Estimated Glomerular Filtration Rate (eGFR) in an upper semi-circle, and a numeric value for the eGFR in a lower semi-circle. The eGFR physiological parameter is related to the Stage 2 CKD diagnosis. In some configurations, the graphical data of each physiological parameter graphical sub-element provide additional information beyond the quantitative value of the physiological parameters to the HCP. For example, in some configurations the physiological parameter data are displayed using color-coded text or color-coded graphics in a graphical sub-element to indicate if the physiological parameters are in-range or out-of-range for a particular PwD. Using the HbA1c physiological parameter 302a as an example, a green color can indicate an HbA1c value that is considered normal for a healthy individual, a yellow color can indicate an elevated HbA1c for a non-insulin dependent diabetic, and a red color can indicates an even more elevated HbA1c that indicates a need for insulin therapy. In some configurations, the graphical sub-element of a physiological parameter also includes an arrow or other graphical indicator that displays a trend of the physiological parameter over time from the prior patient visit, such as an up or down arrow, which indicates a rising or falling trend the HbA1c level or changes in another relevant physiological parameter. In some configurations, the graphical indicators of physiological parameters include graphs, icons, or other non-text graphical indicators that an HCP can easily interpret to assess the condition of the PwD.

In the timeline view 300, the graphical element 306 is depicted as a rectangular graphical element that includes a graphical indicator of a prescribed treatment option for the diagnosed CKD condition. In the example of FIG. 3, the graphical element 306 includes a graphical depiction of a list of CKD treatment options including medications for blood pressure and cholesterol, which are known to affect the progression of CKD, along with options for o quit smoking and changes to diet and exercise for the PwD. During operation, the CDS processor 104 identifies prescribed treatment options based on the diagnosed condition for the PwD along with other relevant PwD medical data 112, and identifies options for prescribed treatments from the diagnostic database 110. For example, in the FIG. 3 the prescribed treatment for the PwD to quit smoking is generated only for a PwD who has a history of smoking as recorded in the PwD medical data 112. The graphical element 306 is also referred to as a trigger graphical element for a prescribed treatment because the HCP optionally selects the graphical element 306 to trigger one or more recommended treatment options. In one embodiment, one or more of the prescribed treatment options are displayed using clickable hyperlinks or with an associated graphical control button to enable the HCP to select a prescribed treatment option. The CDS system 102 then generates a popup window or user interface screen (not shown) to enable the HCP to implement the selected prescribed treatment option, such as prescribing medications or enrolling the PwD in a smoking cessation or diet and exercise program.

Referring again to FIG. 2, the process 200 continues as the CDS processor 104 generates one or more predicted diagnoses for a future condition of the patient based at least in part upon the first diagnosis for the current patient visit, and the predictive model 116 stored in the CDS memory 106 (block 216). As described above, in a Markov Chain, Markov Decision Process, or variants thereof, the CDS processor 102 uses the diagnosis to identify a current state in the predictive model corresponding the diagnosis of the PwD. The CDS processor 102 identifies any states that are linked to the current state in the model as predicted diagnoses, with the transition probabilities in the predictive model corresponding to the likelihood of each predicted diagnosis. For some predicted diagnoses, the CDS processor 102 also uses a prescribed treatment option in addition to the first diagnosed condition for the PwD from the current patient visit to identify predicted diagnoses and associated probability values for the predicted diagnoses in the predictive model. For example, in the example of FIG. 3 the CDS processor 102 generates predicted diagnoses for the PwD if no additional prescribed treatment is given to the PwD to establish a baseline prediction for disease progression, such as CKD progression in the PwD. The CDS processor 102 also uses the recommended prescribed treatment from the diagnostic database 110 to identify a different current state for the PwD in a Markov Chain or as an action input in addition to the current state of the PwD in a Markov Decision Process. In at least some instances, the prescribed treatment for the PwD affects the probability values for future predicted diagnoses, and the predictive model 116 returns a set of one or more predicted diagnoses with different corresponding probability values in comparison to a baseline prediction for disease progression with no treatment. In some instances, the predictive model generates multiple sets of predicted diagnoses for multiple different prescribed treatment options. Furthermore, in some embodiments the CDS processor 104 provides additional medical data from the PwD medical data 112 as inputs to the predictive model 116 to refine the predicted diagnosis outcomes. For example, demographic and other relevant medical risk factor data for the PwD (e.g. if the PwD smokes) beyond the currently diagnosed condition can be provided to the predictive model 116 to generate the predicted diagnoses. Additionally, in embodiments of the predictive model 116 that use a semi-Markov Chain or semi-Markov Decision Process, the CDS processor 102 also provides data corresponding to the duration of how long the PwD has remained stable in diagnosis of the current patient visit as a “sojourn time” to the predictive model 116.

The process 200 continues as the CDS processor 104 generates graphical elements for one or more of the predicted diagnoses based on a ranking of probabilities for the predicted diagnoses that are returned from the predictive model 116 (block 220). In the illustrative embodiment of FIG. 2, the CDS processor 104 identifies the predicted diagnosis with the highest probability as the highest-ranked predicted diagnosis for each potential prescribed treatment option, such as the CKD treatment option and the no-additional-treatment baseline option that are described above. As described below, during operation an HCP may provide user input to view additional predicted diagnoses with lower probability values to review different potential disease progression outcomes with the PwD. Alternative configurations may employ another ranking process such as a predetermined probability threshold for predicted diagnoses and select any predicted diagnosis that exceeds the predetermined probability threshold. The CDS processor 104 generates the graphical element of each selected predicted diagnosis with a graphical indicator of the predicted diagnosis, and at least one graphical sub-element that is relevant to a physiological parameter that is related to the predicted diagnosis. In particular, the at least one graphical sub-element includes a graphical indicator of a range of values of the physiological parameter that correspond to the predicted diagnosis. The CDS processor 104 generate the graphical element for each predicted diagnosis with least one of a shape, color, or label that is different from a corresponding shape, color, or label of the first graphical element of the currently diagnosed condition to indicate that the graphical element corresponds to a future predicted diagnosis. The distinction between the graphical elements for current or past diagnoses and the graphical elements for predicted diagnoses assists the HCP in quickly assessing information in the timeline view and reduces the likelihood that the HCP misinterprets a predicted diagnosis as an existing diagnosis for the PwD. In the embodiments described herein, the graphical elements of predicted diagnoses include rectangles with rounded corners compared to the right-angle corners of graphical elements for the first diagnosis in the current patient visit, but in alternative embodiments the predicted diagnoses can be distinguished by using different colors, text or graphical icons in labels, or combinations of different shapes, colors, and labels.

Referring to FIG. 3, the timeline view 300 includes the graphical elements 312 and 320 for predicted diagnoses. In the timeline view 300, the graphical elements 312 and 320 for the predicted diagnoses indicate the predicted diagnosis for the condition of the PwD at the time of a future patient visit, which is a 6-month time span in the example of FIG. 3. The graphical element 312 is the predicted diagnosis having the highest probability value in the predictive model 116 in response to performing the prescribed treatment for CKD that is depicted in graphical element 306. The graphical element 312 includes a graphical indicator for the predicted diagnosis, which in this case is “Type 2 Diabetes/Stage 2 CKD”, and a text label “Predicted: Stable” that indicates a prediction that the PwD will remain in the same general condition as is diagnosed in the current patient visit. The graphical element 312 also contains graphical sub-elements 314a and 314b of estimated ranges of relevant physiological parameters that are related to the predicted diagnosis. The graphical sub-element 314a includes a graphical depiction of an estimated range of HbA1c and the graphical sub-element 314b includes a graphical depiction of an estimated range of eGFR. In FIG. 3, the graphical element 320 is the predicted diagnosis having the highest probability value in the predictive model 116 in response to performing no additional treatments. The graphical element 320 includes a graphical indicator for the predicted diagnosis, which in this case is “Type 2 Diabetes/Stage 3 CKD”, and a text label “Predicted: Progression” that indicates a prediction that the PwD will experience a progression in a disease condition from the diagnosed condition in the current patient visit. The graphical element 320 also contains graphical sub-elements 322a and 322b of estimated ranges of values for relevant physiological parameters that are related to the predicted diagnosis. The graphical sub-element 322a includes a graphical depiction of an estimated range of HbA1c and the graphical sub-element 322b includes a graphical depiction of an estimated range of eGFR, which is a different range from the predicted value in graphical sub-element 314a due to the prediction of a progression of the CKD condition for the PwD. In the examples disclosed herein, the graphical sub-elements of predicted physiological parameters include ranges of multiple estimated values for relevant physiological parameters, but in alternative configurations the range is instead a single predicted physiological parameter value. As described above, the graphical elements 312 and 320 include a rectangular shape with a rounded corner shape that is distinct from the right-angle shape of the graphical element 302 for the diagnosis in the current patient visit. Additionally, the text labels in the graphical elements 312 and 320 indicated that the diagnoses are a prediction.

Referring again to FIG. 2, the process 200 continues as the CDS processor 104 generates graphical data corresponding to a timeline view that includes the graphical elements that are described above with reference to the processing of blocks 212-220 as well as graphical connectors that connect these graphical elements and a timeline slider that enables dynamic adjustment of the timeline view (block 224). In addition to including an arrangement of the previously generated graphical elements, the timeline view generates one or more graphical element that depict a time range over which the patient visits and prescribed therapies occur. The timeline view further includes a timeline slider that enables an HCP to adjust both the size of the time range that is depicted in the timeline view and to move the time range depicted in the timeline view forward and backward in time.

Upon generation of the graphical data corresponding to the timeline view in block 224 of the process 200, the CDS system 102 uses the CDS processor 104 and network transceiver 118 to transmit the generated graphical data for the timeline view user interface to the client HCP terminal 128 via the network 148. The HCP terminal 128 receives the graphical data and the terminal processor 130 executes the terminal software 134, such as a web browser or other client software, to generate a rendered user interface with a visual depiction of the timeline view using the display device 136 that is provided in the HCP terminal 128. The HCP interacts with the user interface using the HCP terminal 128, and as described below the HCP optionally provides input to the HCP terminal to update the timeline user interface, which the CDS system 102 receives via the network 148 and processes to provide an updated set of graphical data for an updated timeline view to the HCP terminal 128. Additionally, in some configurations the CDS system 102 optionally transmits the generated graphical data for the timeline view user interface to the PWD device 138 for direct display to the PwD using the display device 146. While the system 100 is embodied as a networked system in which the CDS system 102 is connected to the HCP terminal 128 via the network 148 for illustrative purposes, in another configuration a single computing system performs the operations of both the CDS system 102 and the HCP terminal 128. In this configuration, a processor in a single computing system generates the graphical data corresponding to the timeline view of the patient visit and operates a display device that is provided in the single computing system to display the graphical data corresponding to the timeline view of the patient visits. In one configuration, the single computing system is the HCP terminal 128 that is further reconfigured to host the CDS software 108, diagnostic database 110, PwD data 112, graphics data 114, and predictive model 116 in addition to the terminal software 134.

Referring again to FIG. 3, the timeline view 300 includes the previously mentioned graphical elements 302, 306, 312, and 320 arranged linearly along a timeline that is depicted with a timeline graphical element 324. The timeline graphical element 324 includes date information pertaining to the time of the current patient visit (September 2021) and a scheduled time of a future patient visit that corresponds to the time range for the predicted diagnoses (March 2022). A timeline slider graphical element 326 in the timeline view graphical element 324 enables an HCP to adjust the time range that is depicted in the timeline view 300. While not described in further detail herein, the timeline slider 326 enables the HCP to review previous patient visits and older medical history for the PwD based on the PwD medical data 112, and, as described in further detail below, the timeline slider 326 enables the HCP to view predicted diagnoses for additional future patient visits. Graphical connectors indicate the relationships between the graphical element 302 of the current patient visit, the prescribed treatment trigger graphical element 306, and the predicted diagnosis graphical elements 312 and 320. In FIG. 3, the graphical connectors are formed as linear straight or curved connectors with arrows that indicate the flow of time in the timeline view 300. In particular, a graphical connector formed from graphical sub-connectors 308a and 308b connects the current patient visit diagnosis graphical element 302 to the predicted diagnosis graphical element 312 via the prescribed treatment graphical element 306. The graphical sub-connectors 308a and 308b link the current diagnosis, prescribed treatment, and predicted future diagnosis with an easily understandable structure for the HCP. Another graphical connector 316 links the current patient visit diagnosis graphical element 302 with the predicted diagnosis graphical element 320, which indicates the highest probability predicted diagnosis for the PwD if the PwD receives no additional treatment. The timeline view 300 also includes a graphical indication of the probability for each predicted diagnosis that is associated with a corresponding graphical connector in the timeline view. For example, an expected probability value 310 is positioned in association with the graphical sub-connector 308a or 308b (308b in the example of FIG. 3) and another expected probability value 318 is associated with the graphical connector 316. The expected probability values are the predicted probabilities for reaching the corresponding predicted diagnoses that are generated from the predictive model 116. In the example of FIG. 3, the expected probability values are graphical indicators displayed as numeric values in a range of 0.0 to 1.0, but in other embodiments the expected probability values are displayed as percentages, as ranked values (e.g. first, second, third most likely), or qualitatively such as adjusting the thickness or color of the graphical connector lines to indicate the likelihood of a particular predicted diagnosis. FIG. 3 also depicts a ranking selector widget 330, which is embodied as a drop-down selector input in the timeline view 300. The ranking selector widget enables an HCP or other user to adjust the ranking threshold for the predicted diagnoses that are displayed in the timeline view, and in the illustrative example of FIG. 3 the default rank is to display only the highest-ranked (1) predicted diagnoses in the timeline view 300.

Those of skill in the art will recognize that the processing of steps 212-220 may occur in a different order than what is described above or concurrently. Additionally, the timeline views depicted herein are arranged in a left-to-right format representing the present time of a patient visit to future times for predicted diagnoses, but alternative configurations can orient the timeline in right-to-left format or vertically in a top-to-bottom or bottom-to-top format. Additionally, the alternative configurations of the process 200 can include different visual formats and arrangements of the graphical elements, graphical sub-elements, and graphical connectors that are depicted herein.

Referring again to FIG. 2, during the process 200 the HCP has the option to modify the timeline view to adjust the timeline range (block 228), to adjust the ranking threshold (block 240), and to adjust both the timeline range and the ranking threshold. In particular, the time range for the predicted diagnoses extends to the next patient visit in the default timeline view of FIG. 3, and the HCP has the option to extend the time range to cover one or more future patient visits. To adjust the timeline range the HCP operates the HCP terminal 128 to select the timeline slider 326 and pull the timeline slider to the right with a click-and-drag operation or other suitable user input. The CDS processor 104 uses the predictive model 116 to identify additional predicted diagnoses for the future time range in a similar manner to that described in the processing of block 216 using the existing predicted diagnoses and other estimated data as inputs to the predictive model (block 232). The CDS processor 104 generates updated graphical data in the timeline view including additional graphical elements for additional predicted diagnoses in future patient visits (block 236), and the HCP terminal 128 or other client computing device displays the updated timeline view.

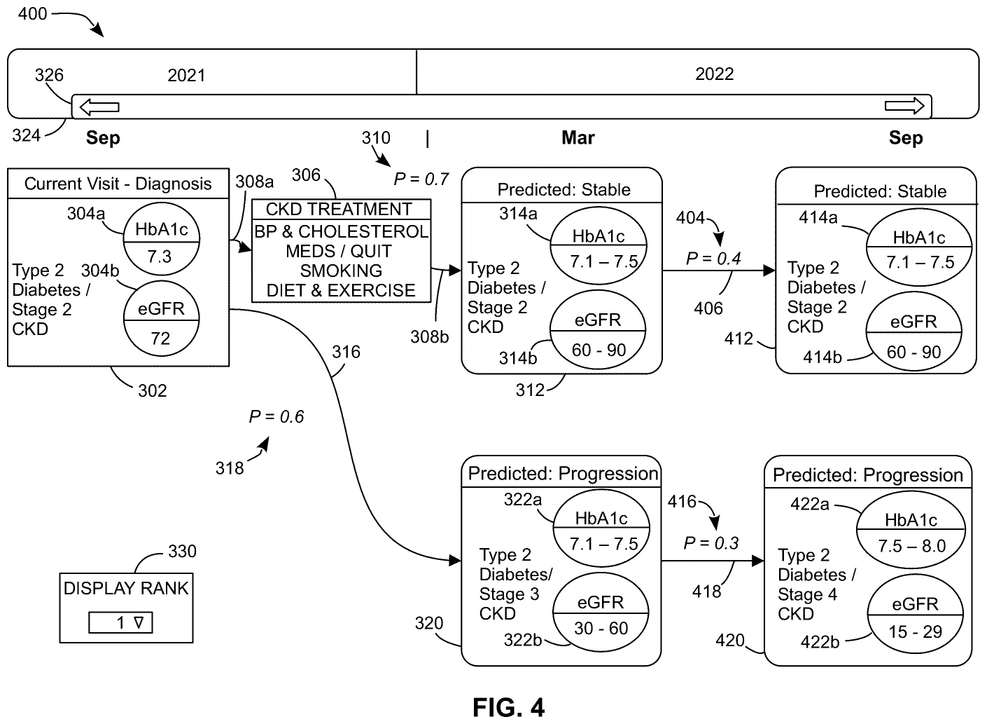

FIG. 4 depicts an example of an expanded timeline view 400 that the CDS system 102 generates in response to an input to display predicted diagnoses for two future patient visits. The timeline view 400 includes the elements of the timeline view 300 with the timeline graphical element 324 now displaying a larger time range that includes a second future patient visit in September 2022. In the example of FIG. 4, a graphical element 412 represents a second predicted diagnosis for a stable condition of the PwD if the PwD receives and continues the prescribed treatment for CKD that is depicted in the graphical element 306. The graphical sub-elements 414a and 414b depict the estimated HbA1c and eGFR physiological parameter range value estimates, respectively. A graphical connector 406 depicts the progression from the predicted diagnosis in graphical element 312 and 412, with an expected probability indicator 404 depicting the likelihood of the predicted diagnosis 412 along the given treatment path. Similarly, the graphical element 420 depicts a second predicted diagnosis of a continued progression of diabetes with stage 3 CKD if the PwD receives no further treatment. The graphical element 420 includes a prediction of a range of further elevated HbA1c levels in a graphical sub-element 422a and a range of further depressed eGFR levels in a graphical sub-element 422b. A graphical connector 418 connects the earlier predicted diagnosis in graphical element 320 to the graphical element 420, with an expected probability indicator 416 depicting the likelihood of the predicted diagnosis 420 along the given treatment path.

Referring again to FIG. 2, to adjust the ranking threshold (block 240) the HCP terminal 128 receives in input from the HCP via the ranking selector widget 330 to adjust the ranking threshold for the number of predicted diagnoses to display in the timeline view. While the ranking selector widget 330 depicted herein selects based on a numerical rank, in an alternative configuration the ranking threshold is a minimum probability value corresponding to the likelihood of a given predicted diagnosis in the predictive model 116. The CDS system 102 receives the updated ranking threshold data, the CDS processor 104 uses the predictive model 116 to identify all predicted diagnoses that meet the updated ranking threshold, and the CDS processor 104 generates updated graphical data of the timeline view including graphical elements for newly added predicted diagnoses or, in instances where the ranking threshold is raised, potentially removing graphical elements for predicted diagnoses with expected probability values that are less than the new ranking threshold. The CDS processor 104 generates the graphical data of the updated timeline view for the predicted diagnoses with expected probabilities that exceed the ranking threshold (block 244), and the HCP terminal 128 or other client computing device displays the updated timeline view.

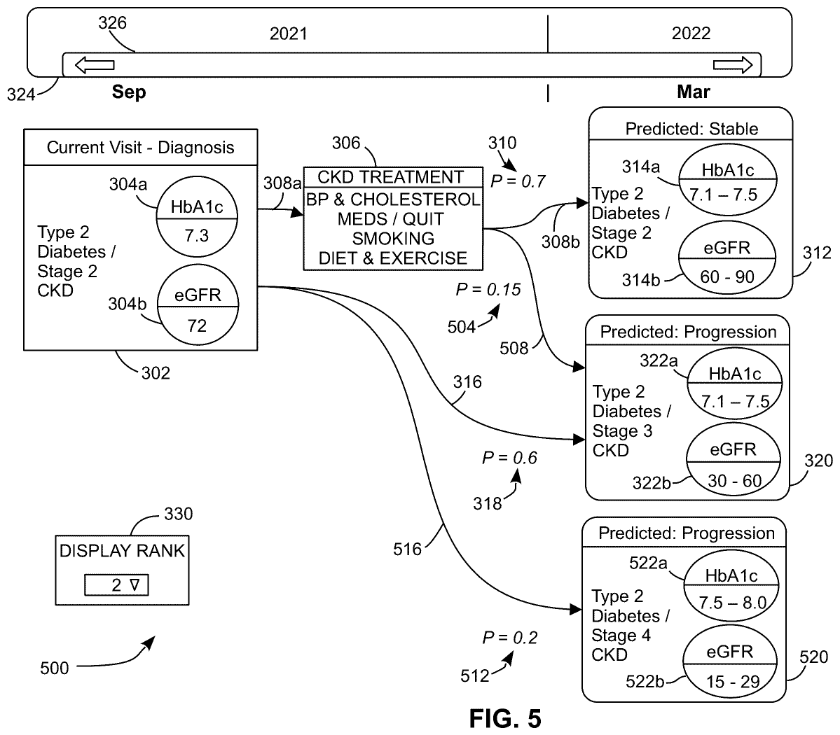

FIG. 5 depicts an example of a timeline view 500 that the CDS system 102 generates in response to an input to adjust the ranking threshold to show the predicted diagnoses having both the highest probability and second highest probability in the predictive model 116. The timeline view 500 includes the graphical elements of FIG. 3 along with an additional graphical element 520 for a newly generated predicted diagnosis and additional connector graphical elements 508 and 516 with corresponding expected probability graphical indicators 504 and 512. Additionally, the ranking selector widget 330 displays a “2” to indicate that predicted diagnoses with the highest and second highest (first and second ranked) expected probabilities in the predictive model 116 are included in the timeline view 500. In more detail, the graphical element 520 depicts another predicted diagnosis in which the PwD experiences a progression of diabetes with further elevated HbA1c levels and an accelerated progression to stage 4 CKD with further depressed eGFR levels by the time of the next scheduled patient visit if the PwD does not receive treatment for the CKD diagnosis. The graphical element 520 includes the graphical sub-elements 522a and 522b that depict the estimated ranges of HbA1c and eGFR, respectively. FIG. 5 also includes a new graphical sub-connector 508 that connects the graphical element 306 to the graphical element 320, which indicates that there is some probability for a progression to stage 3 CKD even if the PwD undergoes the recommended CKD treatment. The timeline view 300 displays both predicted branches with the graphical sub-connector 308b and associated graphical indicator of expected probability value 310 along with the sub-connector 508 with the graphical indicator of the lower expected probability value 504. Similarly, the graphical connector 516 connects the graphical element 302 for the diagnosis in the current patient visit to the graphical element 520 of the third predicted diagnosis. The graphical indicator of the expected probability 512 displays the expected probability value that the PwD will reach the advanced CKD stage, which is a lower probability value than the graphical indicator of the expected probability 318 for the branched graphical connector 316. As such, the process 200 as exemplified in the timeline view 500 enables the HCP to view greater or lesser numbers of predicted diagnoses, and generates a display of how different treatment paths may lead to diverging or converging predicted diagnosed conditions.

The embodiments described herein enable the generation of timeline view user interfaces that present highly relevant diagnosis, predicted diagnosis, physiological parameter, and recommended prescribed treatment options to an HCP. The user interface reduces the cognitive burden on the HCP to enable more efficient and effective treatment for PwDs and other patients. When integrated within a clinical workflow, the embodiments described herein can help HCPs in making improved personalized therapy decisions improving clinical, patient-reported and economical outcomes. In particular, the embodiments described herein enable an HCP to work with existing clinical guidelines for therapy transitions in diabetes in an efficient manner, because these guidelines may be cumbersome to follow or apply given the volume of patient data in prior-art systems. In addition, the embodiments described herein enable the HCP to review one or more potential future diagnoses that are likely to result from various prescribed treatment options, which enables the HCP to analyze and discuss different treatment options with the PwD in an efficient and effective manner.

This disclosure is described in connection with what are considered to be the most practical and preferred embodiments. However, these descriptions are presented by way of illustration and are not intended to be limited to the disclosed embodiments. Accordingly, one of skill in the art will realize that this disclosure encompasses all modifications and alternative arrangements within the spirit and scope of the disclosure and as set forth in the following claims.

What is claimed is:

1. A method for generating a user interface of predicted disease progressions for a patient comprising:
receiving, with a processor, medical data for the patient, the medical data corresponding to at least one patient visit to a healthcare provider;
generating, with the processor, a first diagnosis for the patient during a current patient visit based on the medical data;
generating, with the processor, a first predicted diagnosis for a future condition of the patient based at least in part upon the first diagnosis and a predictive model stored in a memory operatively connected to the processor; and
generating, with the processor, graphical data corresponding to a timeline view of the current patient visit and the first predicted diagnosis, the generating of the graphical data further comprising:
generating a first graphical element corresponding to the first diagnosis, the first graphical element further comprising:
a graphical indicator of the first diagnosis; and
at least one graphical sub-element, the at least one graphical sub-element being relevant to a physiological parameter selected from the medical data, the physiological parameter being related to the first diagnosis; and
generating a second graphical element corresponding to the first predicted diagnosis, the second graphical element further comprising:
a graphical indicator of the first predicted diagnosis; and
at least one graphical sub-element, the at least one graphical sub-element being relevant to a physiological parameter related to the first predicted diagnosis; and
generating a first graphical connector between the first graphical element and the second graphical element, the first graphical connector indicating a progression of time between a first time of the current patient visit and a second time in the timeline view.

2. The method of claim 1 further comprising:
generating, with the processor, a second predicted diagnosis for the future condition of the patient based at least in part upon the first diagnosis, a recommended prescribed treatment, and the predictive model; and
the generating, with the processor, of the graphical data corresponding to the timeline view further comprising:
generating a third graphical element including a graphical indicator of the recommended prescribed treatment;
generating a fourth graphical element corresponding to the second predicted diagnosis, the fourth graphical element further comprising:
a graphical indicator of the second predicted diagnosis; and
at least one graphical sub-element, the at least one graphical sub-element being relevant to a physiological parameter related to the second predicted diagnosis; and
generating a second graphical connector between the first graphical element and the fourth graphical element, the second graphical connector indicating a progression of time between the first time of the current patient visit and a third time in the timeline view.

3. The method of claim 2, wherein the second connector further comprises:
a first sub-connector that connects the first graphical element to the third graphical element and a second sub-connector that connects the third graphical element to the fourth graphical element.

4. The method of claim 2, the generating of the second graphical connector further comprising:
generating, with the processor, a graphical indicator of an expected probability for the second predicted diagnosis that is associated with the second graphical connector.

5. The method of claim 1, wherein the processor generates the second graphical element with at least one of a shape, color, or label that is different from a corresponding shape, color, or label of the first graphical element to indicate that the second graphical element corresponds to a future predicted diagnosis.

6. The method of claim 1, the generating of the first graphical connector further comprising:
generating, with the processor, a graphical indicator of an expected probability for the first predicted diagnosis that is associated with the first graphical connector.

7. The method of claim 1 further comprising:
generating, with the processor, a second predicted diagnosis for a future condition of the patient based at least in part upon the first predicted diagnosis, and the predictive model; and
the generating, with the processor, of the graphical data corresponding to the timeline view further comprising:
generating graphical data corresponding to a timeline slider in the timeline view;
generating a third graphical element relevant to the second predicted diagnosis in the medical data during a third time, the third time occurring after the second time, in response to a user input to the timeline slider that moves to the third time in the timeline view, the third graphical element further comprising:
a graphical indicator of the second predicted diagnosis; and
at least one graphical sub-element, the at least one graphical sub-element being relevant to a physiological parameter related to the second predicted diagnosis; and
generating a second graphical connector between the second graphical element and the third graphical element, the second graphical connector indicating a progression of time between the second time of the first predicted diagnosis and the third time of the second predicted diagnosis in the timeline view.

8. The method of claim 7 further comprising:
generating, with the processor, a graphical indicator of an expected probability for the second predicted diagnosis that is associated with the second graphical connector.

9. The method of claim 1 further comprising:
generating, with the processor, the first predicted diagnosis for the future condition of the patient based at least in part upon the first diagnosis and the predictive model, wherein the first predicted diagnosis has a highest probability in the predictive model;
generating, with the processor, a second predicted diagnosis for the future condition of the patient based at least in part upon the first diagnosis and the predictive model, wherein the second predicted diagnosis has a second highest probability in the predictive model; and
the generating, with the processor, of the graphical data corresponding to the timeline view further comprising:
generating a third graphical element corresponding to the second predicted diagnosis, the second graphical element further comprising:
a graphical indicator of the second predicted diagnosis; and
at least one graphical sub-element, the at least one graphical sub-element being relevant to a physiological parameter related to the second predicted diagnosis; and
generating a second graphical connector between the first graphical element and the third graphical element, the second graphical connector indicating a progression of time between a first time of the current patient visit and a second time in the timeline view.

10. A computing system configured to generate a user interface of a treatment history for a patient comprising:
a memory configured to store:
medical data for the patient, the medical data corresponding to at least one patient visit to a healthcare provider;
a predictive model; and
stored program instructions; and
a processor operatively connected to the memory, the processor being configured to execute the stored program instructions to:
generate a first diagnosis for the patient during a current patient visit based on the medical data;
generate a first predicted diagnosis for a future condition of the patient based at least in part upon the first diagnosis and the predictive model; and
generate graphical data corresponding to a timeline view of the current patient visit and the first predicted diagnosis, the processor being further configured to:
generate a first graphical element corresponding to the first diagnosis, the first graphical element further comprising:
a graphical indicator of the first diagnosis; and
at least one graphical sub-element, the at least one graphical sub-element being relevant to a physiological parameter selected from the medical data, the physiological parameter being related to the first diagnosis; and
generate a second graphical element corresponding to the first predicted diagnosis, the second graphical element further comprising:
a graphical indicator of the first predicted diagnosis; and
at least one graphical sub-element, the at least one graphical sub-element being relevant to a physiological parameter related to the first predicted diagnosis; and
generate a first graphical connector between the first graphical element and the second graphical element, the first graphical connector indicating a progression of time between a first time of the current patient visit and a second time in the timeline view.

11. The computing system of claim 10, the processor being further configured to:
generate a second predicted diagnosis for the future condition of the patient based at least in part upon the first diagnosis, a recommended prescribed treatment, and the predictive model; and
generate the graphical data corresponding to the timeline view further comprising:
a third graphical element including a graphical indicator of the recommended prescribed treatment;
a fourth graphical element corresponding to the second predicted diagnosis, the fourth graphical element further comprising:
a graphical indicator of the second predicted diagnosis; and
at least one graphical sub-element, the at least one graphical sub-element being relevant to a physiological parameter related to the second predicted diagnosis; and
a second graphical connector between the first graphical element and the fourth graphical element, the second graphical connector indicating a progression of time between the first time of the current patient visit and a third time in the timeline view.

12. The computing system of claim 11, the processor being further configured to:
generate the second graphical connector further comprising a first sub-connector that connects the first graphical element to the third graphical element and a second sub-connector that connects the third graphical element to the fourth graphical element.

13. The computing system of claim 11, the processor being further configured to:
generate a graphical indicator of an expected probability for the second predicted diagnosis that is associated with the second graphical connector.

14. The computing system of claim 10, wherein the processor is configured to generate the second graphical element with at least one of a shape, color, or label that is different from a corresponding shape, color, or label of the first graphical element to indicate that the second graphical element corresponds to a future predicted diagnosis.

15. The computing system of claim 10, the processor being further configured to:
generate a graphical indicator of an expected probability for the first predicted diagnosis that is associated with the first graphical connector.

16. The computing system of claim 10, the processor being further configured to:
generate a second predicted diagnosis for a future condition of the patient based at least in part upon the first predicted diagnosis and the predictive model; and
generate the graphical data corresponding to the timeline view further comprising:
graphical data corresponding to a timeline slider in the timeline view;
a third graphical element relevant to the second predicted diagnosis in the medical data during a third time, the third time occurring after the second time, in response to a user input to the timeline slider that moves to the third time in the timeline view, the third graphical element further comprising:
a graphical indicator of the second predicted diagnosis; and
at least one graphical sub-element, the at least one graphical sub-element being relevant to a physiological parameter related to the second predicted diagnosis; and
a second graphical connector between the second graphical element and the third graphical element, the second graphical connector indicating a progression of time between the second time of the first predicted diagnosis and the third time of the second predicted diagnosis in the timeline view.

17. The computing system of claim 16, the processor being further configured to:
generate a graphical indicator of an expected probability for the second predicted diagnosis that is associated with the second graphical connector.

18. The computing system of claim 10, the processor being further configured to:
generate the first predicted diagnosis for the future condition of the patient based at least in part upon the first diagnosis, and the predictive model, wherein the first predicted diagnosis has a highest probability in the predictive model;
generate a second predicted diagnosis for the future condition of the patient based at least in part upon the first diagnosis, and the predictive model, wherein the second predicted diagnosis has a second highest probability in the predictive model; and
generate the graphical data corresponding to the timeline view further comprising:
a third graphical element corresponding to the second predicted diagnosis, the second graphical element further comprising:
a graphical indicator of the second predicted diagnosis; and
at least one graphical sub-element, the at least one graphical sub-element being relevant to a physiological parameter related to the second predicted diagnosis; and
a second graphical connector between the first graphical element and the third graphical element, the second graphical connector indicating a progression of time between a first time of the current patient visit and a second time in the timeline view.

19. The computing system of claim 10, further comprising:
a network transceiver; and
the processor being operatively connected to the network transceiver and further configured to:
generate the graphical data corresponding to the timeline view with the processor being provided in a server computing system; and
transmit, with the network transceiver, the graphical data corresponding to the timeline view to a client computing system for display with a display device provided in the client computing system.

20. The computing system of claim 10, further comprising:
a display device; and
the processor being operatively connected to the display device and further configured to:
display the graphical data corresponding to the timeline view with the display device.
